(19) United States
(12) Patent Application Publication
(10) Pub. No.: US 2010/0034794 A1
(43) Pub. Date: Feb. 11, 2010

(54) ENDOTHELIAL PROGENITOR CELL COMPOSITIONS AND NEOVASCULARIZATION

(76) Inventors: Barry W.A. van der Strate, Slochteren (NL); Guido Krenning, Groningen (NL); Brian C.A. Fernandes, Roseville, MN (US); Martin Harmsen, Paterswolde (NL); Marja J.A. van Luyn, Groningen (NL); Didier Billy, Maastricht (FR); Marc Hendriks, Brunssum (NL)

Correspondence Address: K&L Gates LLP, 1900 MAIN STREET, SUITE 600, IRVINE, CA, 92614-7319, US

(21) Appl. No.: 12/443,951

(22) PCT Filed: Oct. 2, 2007
(86) PCT No.: PCT/US07/80224

§ 371 Date: Apr. 1, 2009

Related U.S. Application Data
(60) Provisional application No. 60/828,030, filed on Oct. 3, 2006.

(51) Int. Cl. A61K 35/12

(52) U.S. Cl. 424/93.71

(57) ABSTRACT

Methods and compositions are provided for inducing neovascularization in injured tissues with endothelial progenitor cells (EPCs). Mixtures of purified CD34+ endothelial progenitors and purified CD14+ monocytes, or products of an in vitro co-culture of purified CD34+ endothelial progenitor cells and purified CD14+ monocytes provide neovascularization after administration to a subject having a tissue injury, such as an ischemic injury.

### FIELD OF THE INVENTION

The present invention provides methods and compositions related to induction of neovascularization with endothelial progenitor cells (EPCs) and monocytes in specified compositions for the treatment of injured or diseased tissue.

### BACKGROUND OF THE INVENTION

The development of new blood vessels in response to tissue ischemia constitutes a natural host reaction intended to maintain tissue perfusion required for physiologic organ function. This natural angiogenesis is impaired in advanced age, diabetes and hypercholesterolemia. In each of these conditions, there is a reduction in endogenous expression of vascular endothelial growth factor (VEGF) and exogenous VEGF administration leads to enhanced neovascularization.

Ischemic tissue injury triggers a series of events, including mobilization and recruitment of endothelial progenitor cells (EPCs) to the injury site. In models of post-ischemic angiogenesis, these EPC incorporate into neovessels. Moreover, in animal models, as well as in clinical settings of acute myocardial infarction (aMI), systemic administration of EPC contributes to revascularization of the myocardium and is associated with improved myocardial function.

Since their original description, bone marrow-derived EPC have become a focal point in regenerative therapy following evolving vascular damage. Because numbers of EPC, which are normally low in peripheral blood, increase significantly after an ischemic event, a causal link between vascular damage and EPC-mediated repair has been postulated. In animal models of angiogenesis following ischemia, bone marrow-derived EPC incorporate into neovessels. Moreover, local and systemic levels of angiogenic growth factors, including VEGF, rise after ischemia and are associated with increased numbers of circulating EPC.

The obvious therapeutic potential of exogenous growth factor administration has been successfully assessed in animals and humans. In various animal models, mobilization of EPC after vascular damage by administration of VEGF, granulocyte macrophage colony stimulating factor (GM-CSF), granulocyte colony stimulating factor (G-CSF), fibroblast growth factor 1 (FGF-1), stromal derived factor 1 (SDF-1) or a statin drug, positively correlated with increased numbers of circulating EPC and improved therapeutic neovascularization. Direct evidence for the vasculogenic potential of EPC has been provided by studies in which EPC transplanted in mice with hind limb ischemia incorporated into newly formed blood vessels (Kalka C. et al., “Transplantation of ex vivo expanded EPCs for therapeutic neovascularization,” Proc. Natl. Acad. Sci. 97:3422-7, 2000). In a murine model of myocardial infarction (Ml) intravenous injection of human CD34⁺ EPC contributed to revascularization of the myocardium, and was associated with salvage of myocardial function (Kocher A. A. et al., “Neovascularization of ischemic myocardium by human bone marrow-derived angioblasts prevents cardiomyocyte apoptosis, reduces remodeling and improves cardiac function,” Nat. Med. 7:412-3, 2001). Moreover, intracoronary infusion of autologous EPC into the infarct artery in patients with aMI resulted in increased myocardial viability in the infarct area (Assmus B. et al., “Transplantation of progenitor cells and regeneration enhancement in acute myocardial infarction [TOPCARE-AMI],” Circulation 106:3009-17, 2002).

Current progenitor cell research is focused on the clinical application of EPC in therapeutic neovascularization. Therefore, future large-scale therapeutic application of EPC will require a source of a large numbers of cells.

However, EPCs represent a very small subset of peripheral blood cells, only 0.02% to 0.1% of the peripheral blood mononuclear cells. Thus, in order to obtain a sufficient number of cells for therapeutic treatment, methods for obtaining larger numbers of the appropriate progenitor cells must be developed. Bone-marrow-derived CD34+ or CD133+ EPCs can be mobilized in vivo by administration of granulocyte colon-stimulating factor (G-CSF), however this treatment is associated with a high risk of side effects in patients with vascular diseases. Furthermore, CD34+ cells are difficult to expand in culture. Therefore methods of obtaining large numbers of EPCs suitable for therapeutic neovascularization of injured or diseased tissue are needed.

### SUMMARY OF THE INVENTION

The present invention describes methods for the isolation of human peripheral blood endothelial progenitor cells (EPCs) and human monocytes, the subsequent mixture of the two cell populations in specific combinations, and use of compositions of these mixtures in patients, to generate endothelial cells, or to form new blood vessels or to perform a paracrine function involving the secretion of bioactive factors.

In one embodiment of the present invention, a method is provided for inducing neovascularization in injured tissue in a subject comprising administering to a treatment site in a subject a therapeutically effective amount of a composition comprising a mixture of purified CD34+ endothelial progenitor cells (EPCs) and purified CD14+ monocytes, wherein the administering of the composition to a treatment site results in neovascularization of the injured tissue at the treatment site.

In another embodiment, the CD34+ EPCs are isolated from peripheral blood. In another embodiment, the CD14+ monocytes are isolated from peripheral blood. In yet another embodiment, the CD34+ EPCs and the CD14+ monocytes are autologous.

In another embodiment of the present invention, the CD34+ cells and the CD14+ cells are administered at a ratio of between about 1:1 to about 1,000. In yet another embodiment, the ratio is about 1:100.

In an embodiment of the present invention, the composition further comprises at least one pharmaceutically acceptable carrier which can be a scaffolding material or a biocompatible solution. In another embodiment, the composition further comprises at least one bioactive agent selected from the group consisting of growth factors, chemokines, drugs and cytokines. In yet another embodiment, the at least one growth factor is selected from the group consisting of vascular endothelial growth factor, basic fibroblast growth factor, and combinations thereof.

In another embodiment of the present invention, the therapeutically effective amount of the composition comprises a minimum number of cells necessary for inducing neovascularization in injured cardiac tissue. In another embodiment, the therapeutically effective amount comprises a total of between about 10⁴ and about 10⁸ cells.

In yet another embodiment, the administering step comprises delivery of the composition to the treatment site by a method selected from the group consisting of intra-arterial infusion, intramuscular infusion, intracardiac infusion, intracoronary infusion, intravenous infusion, and combinations thereof. In another embodiment, the injured tissue is ischemic tissue. In yet another embodiment, the ischemic tissue is cardiac tissue.

In one embodiment of the present invention, a method is provided for inducing neovascularization in injured tissue in a subject comprising obtaining peripheral blood from said subject; selecting CD34+ EPCs from said peripheral blood to generate purified CD34+ cells; selecting CD14+ monocytes from said peripheral blood to generate purified CD14+ monocytes; culturing said purified CD34+ cells and said purified CD14+ cells in a culture medium for up to four weeks to yield a population of co-cultured EPCs; and administering a therapeutically effective amount of said co-cultured EPCs to a treatment site in the injured tissue of said subject, thereby inducing neovascularization of said injured tissue at said treatment site.

In another embodiment, the purified CD34+ EPCs and the purified CD14+ monocytes are autologous. In another embodiment, the purified CD34+ EPCs and the purified CD14+ monocytes are cultured at a ratio of between about 1:1 to about 1:1,000. In yet another embodiment, the ratio is about 1:100.

In an embodiment of the present invention, the co-cultured EPCs further comprise at least one pharmaceutically acceptable carrier which can be a scaffolding material or a biocompatible solution. In another embodiment, the composition further comprises at least one bioactive agent selected from the group consisting of growth factors, chemokines, drugs and cytokines. In yet another embodiment, the at least one growth factor is selected from the group consisting of vascular endothelial growth factor, basic fibroblast growth factor, and combinations thereof.

In another embodiment, the therapeutically effective amount of co-cultured EPCs is a minimum number of cells necessary for inducing neovascularization in injured cardiac tissue. In yet another embodiment, the amount of co-cultured EPCs is a total of between about 10⁴ and about 10⁸ cells.

In another embodiment, the administering step comprises delivery of co-cultured EPCs to a treatment site by a method selected from the group consisting of intra-arterial infusion, intramuscular infusion, intracardiac infusion, intracoronary infusion, intravenous infusion, and combinations thereof. In yet another embodiment, the injured tissue is ischemic tissue. In yet another embodiment, the ischemic tissue is cardiac tissue.

In one embodiment of the present invention, a composition is provided for the induction of neovascularization in a subject, comprising: purified CD34+ EPCs; purified CD14+ monocytes; and at least one pharmaceutically acceptable carrier.

In another embodiment, the purified CD34+ EPCs and purified CD14+ monocytes are isolated from peripheral blood. In another embodiment, the purified CD34+ EPCs and purified CD14+ monocytes are cryopreserved and thawed prior to delivery to a treatment site. In yet another embodiment, the purified CD34+ EPCs and purified CD14+ monocytes are autologous.

In another embodiment, the composition comprises purified CD34+ EPCs and purified CD14+ monocytes at a ratio of between about 1:1 to about 1:1,000. In another embodiment, the ratio is about 1:100.

In another embodiment, the pharmaceutically acceptable carrier is a scaffolding material or a biocompatible solution. In another embodiment, the composition further comprises one or more than one bioactive agent selected from the group consisting of cytokines, chemokines, drugs and growth factors. In yet another embodiment, the growth factor is selected from the group consisting of basic fibroblast growth factor, vascular endothelial growth factor, and combinations thereof.

### BRIEF DESCRIPTION OF THE FIGURES

FIG. 1 depicts the number of spindle-shaped cells after 18 days of culture of CD34+ cells alone, CD14+ cells alone or co-cultivation of CD34+ and CD14+ cells at ratios of 1:1, 1:10, 1:100 and 1:1000 according to the teachings of the present invention.

FIGS. 2A-C depict spindle-shaped cells expressing markers for CD14 and CD31 after four weeks of co-culture of CD34+ EPCs and CD14+ monocytes according to the teachings of the present invention. Spindle shape morphology is an indication of endothelial cell formation. CD14+ cells by themselves do not exhibit spindle shape morphology. FIG. 2A depicts red fluorescent (CM-Dil)-labeled spindle-shaped cells, derived from CD14+ cells in close contact with an unlabeled CD34+ cell. FIG. 2B spindle-shaped cells are CD14-derived as indicated by CM-Dil (red) label (CD14+ positive cells were labeled red prior to culture). FIG. 2C: Co-cultured cells express CD31, an endothelial cell marker, after 28 days of culture.

FIG. 3 depicts a Colony Forming Unit (CFU) containing green labeled CD34+ and red labeled CD14+ cells. Mononuclear cells (MNCs) were isolated according to standard procedures and CD34+ and CD14+ cells were isolated from the MNC fraction. CD34+ cells were labeled green with CFSE dye and CD14+ cells were labeled red with CM-Dil. Both labeled cell fractions were mixed with remaining MNC and plated. This figure demonstrates that the CFUs that are formed (from which EC sprout) are predominantly made up from CD14+ and CD34+ cells. The cells that form EC-like cells (spindles) are predominantly CD14-derived. FIG. 3 suggests that the CD34+ cell which lies in the centre of this CFU, is possibly providing the proper signals for the CD14+ cell to differentiate.

FIGS. 4A-E depict CD34+ and CD14+ cells after mono- or co-culturing according to the teachings of the present invention. FIG. 4A depicts spindle-shaped cells after mono-culture of CD34+ cells for 21 days. FIG. 4B depicts spindle-shaped cells after mono-culture of CD14+ cells for 21 days. FIG. 4C depicts spindle-shaped cells after co-cultivation at a ratio of CD34:CD14 of 1:10. FIG. 4D depicts spindle-shaped cells after co-cultivation at a ratio of CD34:CD14 of 1:100. FIG. 4E depicts spindle-shaped cells after co-cultivation at a ratio of CD34:CD14 of 1:1000.

FIGS. 5A-F depict vascularization of Matrigel® implants containing no cells (bare, FIG. 5A), CD34+ cells alone (FIG. 5B), CD14+ cells alone (FIG. 5C), CD34:CD14 cells at a ratio of 1:10 (FIG. 5D), at a ratio of 1:100 (FIG. 5E) and at a ratio of 1:1000 (FIG. 5F) according to the teachings of the present invention.

FIG. 6 depicts FACS analysis of mono- and co-cultured CD14+ and CD34+ cells according to the teachings of the present invention. Expression of the endothelial cell markers vWF and VE-cadherin are higher in co-cultivated cells than in mono-cultured CD14+ cells.

FIG. 7A depicts expression of pro-angiogenic factors in CD34+ EPCs according to the teachings of the present invention. Reverse transcriptase-polymerase chain reaction (RT-PCR) was performed on CD34+ cells that were cultured for 0, 1 and 2 days, either embedded (+) in Matrigel (MG) or not (−). FIG. 7B depicts the effects of pro-angiogenic factors on formation of colony forming units (CFU) activity of CD14+ cells according to the teachings of the present invention.

FIG. 8 depicts analysis of culture supernatants from mono- and co-cultures of CD34+ and CD14+ cells for growth factors according to the teachings of the present invention.

FIG. 9 depicts vascularization of CD34+-loaded Matrigel® implants 14 days after implantation in nude mice according to the teachings of the present invention. Arrows indicate capillaries. 40× magnification.

FIG. 10 depicts CD34+-loaded Matrigel® implants 14 days after implantation in nude mice according to the teachings of the present invention. FIG. 10A depicts clusters of human cells. 100× magnification. FIG. 10B depicts Matrigel implants (M) encapsulated (C) in tissue containing macrophages and fibroblasts. Nearby blood vessels (*) are quiescent. 40× magnification.

FIG. 11 depicts CD14+-loaded Matrigel® implants 14 days after implantation in nude mice according to the teachings of the present invention. FIG. 11A depicts capillaries formed within the Matrigel® implant. 100× magnification. FIG. 11B depicts the sprouting of vessels in the capsulating tissue. 100× magnification.

FIG. 12 depicts CD14+-loaded Matrigel® implants 14 days after implantation in nude mice according to the teachings of the present invention. FIG. 12A depicts inflammatory cells extravasating from an existing blood vessel into the Matrigel® implant. 40× magnification. FIG. 12B depicts a higher magnification of the extravasating inflammatory cells. 100× magnification.

FIG. 13 depicts CD34+:CD14+=1:10 loaded Matrigel® implants 14 days after implantation in nude mice according to the teachings of the present invention. FIG. 13A depicts a vascularized Matrigel® implant with large, organized vessels. 40× magnification. FIG. 13B depicts function capillaries containing erythrocytes in the lumen within the Matrigel® implant. 100× magnification. FIG. 13C depicts thickened capsulating tissue with high cellularity. 20× magnification. FIG. 13D depicts a mast cell which has penetrated the edge of the Matrigel® implant. 100× magnification.

FIG. 14 depicts CD34+:CD14+=1:100 loaded Matrigel® implants 14 days after implantation in nude mice according to the teachings of the present invention. FIG. 14A depicts a vascularized Matrigel® implant with large vessels in an organized structure. 40× magnification. FIG. 14B depicts tube-like structures within the vascularized Matrigel® implant. 40× magnification. FIG. 14C depicts a cross section of a diverging/sprouting vessel. 100× magnification. FIG. 14D depicts activation of the encapsulation tissue with adhesion of inflammatory cells to the endothelial lining. Mast cells are indicated by black arrow. Large vacuolarized cells (immature mast cells) are indicated by white arrows. 40× magnification.

FIG. 15 depicts CD34+:CD14+=1:1000 loaded Matrigel® implants 14 days after implantation in nude mice according to the teachings of the present invention. FIGS. 15A and 15B depict tube-like structures within the vascularized Matrigel® implant. A capillary is indicated by the arrow. FIG. 15C depicts the Matrigel (M)-encapsulating tissue (C) as quiescent and less mature. 40× magnification.

FIG. 16 depicts semiquantitative scoring of neovascularization after subcutaneous implantation of CD34+, CD14+ and combinations at various ratios 14 days after implantation in nude mice according to the teachings of the present invention.

FIG. 17 depicts double expression of the endothelial markers CD31 and vWF in CD34+/CD14+ co-cultures according to the teachings of the present invention. After three weeks in culture, high percentages of CD31+/vWF+ expression was seen in the co-cultures.

FIG. 18 depicts double expression of the endothelial markers CD144 (VE-cadherin) and eNOS in CD34+/CD14+ co-cultures according to the teachings of the present invention. After three weeks in culture, high percentages of CD144/eNOS expression was seen in the co-cultures.

FIG. 19 depicts expression of CD31/vWF and CD144/eNOS on CD14+ cells alone, that were cultured with supernatant that was obtained from either CD34+ alone, CD14+ alone, or cocultivations of CD34+ with CD14+ cells. This experiment implicates that soluble factors, secreted by CD34+ cells, induce the enhanced endothelial proliferation of the CD14 cells “according to the teachings of the present invention”

FIG. 20 depicts expression of K167 proliferation marker in co-cultured CD34+/CD14+ cells according to the teachings of the present invention.

FIG. 21 depicts prevention of thrombin generation by CD34+/CD14+ co-cultured cells according to the teachings of the present invention. Human umbilical vein endothelial cells (HUVEC) are the positive control and vascular smooth muscle cells (VSMC) are the negative control. ND=not determined.

FIG. 22 depicts the presence of human cells within a Matrigel® implant according to the teachings of the present invention. FIG. 22A depicts a cell within a tube-like structure. FIG. 22B depicts a human cell within a small vessel, suggesting that a human (probably CD14+ cell), differentiated endothelial cell has incorporated into the neovasculature 100× magnification.

FIG. 23 depicts the induction of murine (host) neovascularization in human CD34+/CD14 loaded Matrigel® implants after 14 days according to the teachings of the present invention. FIG. 23A depict a bare Matrigel® control. 20× magnification. FIG. 23B depicts a sprouting capillary expressing murine CD31, the inserts show a single cell capillary (lower left insert) and a cluster (upper right insert) expressing murine CD31. FIG. 23C depicts a large vessel expressing murine CD31. FIG. 23D depicts a longitudinal section through a murine CD31+ vessel with erythrocytes within the vessel lumen. FIG. 23E depicts a large vessel with a longitudinal section (right side of image) and transverse section (arrow).

FIG. 24 depicts the recruitment of murine monocytes/macrophages into human CD14 loaded Matrigel® implants after 14 days according to the teachings of the present invention. 40× magnification.

FIG. 25 depicts the expression of vascular endothelial growth factor (VEGF) in CD34+:CD14+=1:10 loaded Matrigel® implants after 14 days according to the teachings of the present invention. 40× magnification.

### DETAILED DESCRIPTION OF THE INVENTION

Prior to setting forth the invention, it may be helpful to an understanding thereof to set forth definitions of certain terms that will be used hereinafter:

Bioactive agents: As used herein, “bioactive agents” refers to any organic, inorganic, or living agent that is biologically active or relevant. For example, a bioactive material can be a protein, a polypeptide, a polysaccharide (e.g. heparin), an oligosaccharide, a mono- or disaccharide, an organic compound, an organometallic compound, or an inorganic compound. It can include a biologically active molecule such as a hormone, a growth factor, a growth factor producing virus, a growth factor inhibitor, a growth factor receptor, an anti-inflammatory agent, an antimetabolite, an integrin blocker, or a complete or partial functional insense or antisense gene. It can also include a man-made particle or material, which carries a biologically relevant or active agent. Bioactive agents also can include drugs such as chemical or biological compounds that can have a therapeutic effect on a biological organism. Bioactive agents include those that are especially useful for long-term therapy such as hormonal treatment. Examples include drugs for contraception and hormone replacement therapy, and for the treatment of diseases such as osteoporosis, cancer, epilepsy, Parkinson's disease and pain. Suitable biological agents can include, e.g., anti-inflammatory agents, anti-infective agents (e.g., antibiotics and antiviral agents), analgesics and analgesic combinations, antiasthmatic agents, anticonvulsants, antidepressants, antidiabetic agents, antineoplastics, anticancer agents, antipsychotics, and agents used for cardiovascular diseases such as anti-restenosis and anti-coagulant compounds. Bioactive agents can also include growth factors, cytokines, chemokines such as, but not limited to, vascular endothelial growth factor, transforming growth factor beta, insulin growth factor, platelet-derived growth factor, fibroblast growth factor, and combinations thereof.

Biocompatible: As used herein “biocompatible” shall mean any material that does not cause injury or death to the animal or induce an adverse reaction in an animal when placed in intimate contact with the animal's tissues. Adverse reactions include chronic inflammation, infection, excessive fibrotic tissue formation, excessive cell death, or thrombosis.

Co-cultured endothelial progenitor cells: As used herein, “co-cultured endothelial progenitor cells” refers to cells resulting from the co-culture of CD34+ EPCs and CD14+ monocytes at a variety of ratios and culture conditions.

Composition(s): As used herein, “composition(s)” refers to both co-cocultured EPCs and mixtures of CD34+ EPCs and CD14+ monocytes which optionally can additionally contain bioactive agents.

Injury: As used herein, “injury” refers to a tissue damaged by trauma or disease or as a result of the aging process.

Ischemia: As used herein, “ischemia” refers to insufficient blood supply to a specific organ or tissue, usually caused by a blood vessel disease, but can also result from vessel injury, constriction, or inadequate blood flow due to inefficient action of the heart. Specific ischemic conditions include, but are not limited to, limb ischemia, chronic myocardial ischemia, ischemic cardiomyopathy, myocardial ischemia, cerebrovascular ischemia, renal ischemia, pulmonary ischemia and intestinal ischemia.

Neovascularization: As used herein, “neovascularization” refers to the formation of new blood vessels.

Treatment Site: As used herein “treatment site” shall mean a site of tissue injury or disease or a site adjacent to the site of tissue injury of disease.

### DETAILED DESCRIPTION OF THE INVENTION

The present invention provides compositions and methods related to the induction of neovascularization by compositions comprising endothelial progenitor cells (EPCs) or EPC/monocyte mixtures or co-cultures for the treatment of injured tissues. Injured tissues, including damaged cardiac tissue, such as, but not limited to ischemic tissue, can be treated by increasing the blood flow to the tissue. Such increase in blood flow can be mediated, for example, by increasing the number of blood vessels which supply that tissue. The production of blood vessels is accomplished by two main processes: angiogenesis and vasculogenesis. Angiogenesis refers to the production of vascular tissue from fully differentiated endothelial cells derived from pre-existing native blood vessels. Angiogenesis is induced by complex signaling mechanisms of cytokines and growth factors including vascular endothelial growth factor (VEGF), basic fibroblast growth factor (bFGF), and other mediators. This process is mediated by the encroachment of “activated” endothelial cells through the disrupted basement membrane into the interstitium possibly via an ischemic signal.

In contrast, neovasculogenesis, which until recently was believed to occur only in embryos, is the formation of vascular tissues in situ from EPCs. The EPCs can be recruited from the bone marrow or peripheral blood or can be introduced exogenously into a subject.

Neovascularization by EPC has been a topic of intense research during the past decade. The rare CD34+ hematopoietic stem cell is often designated as the archetype EPC, because it can contribute to the repair of vascular damage in vivo.

Monocytes, defined by the CD14 surface marker, have been demonstrated to differentiate towards an endothelial phenotype in vitro and in vivo. Because the CD14+ monocytic cells are by far more frequent in peripheral blood than CD34+ cells (approximately 10% CD14+ vs. 0.01% CD34+) these cells would therefore seem to be appropriate candidates for a cellular contribution to tissue generation and repair. Cultivation of peripheral blood mononuclear cells (PBMNC) revealed co-localization of CD34+ EPC and monocytes at the sites of endothelial cell differentiation in vitro. Therefore an interaction between CD34+ EPC and monocytes can lead to increased endothelialization in vitro.

Over the past several years EPC have become a focal point in cardiovascular regenerative therapy, especially since therapeutic mobilization of EPC by growth factor administration and transplantation of these cells into the infarcted region have proven beneficial for patients with ischemic conditions. However, there is accumulating evidence that EPC are phenotypically and functionally a heterogeneous population with endothelium-forming capacity (see co-pending U.S. patent application Ser. No. 11/202,514 filed Feb. 16, 2006. When isolated by flow cytometry and cultured under angiogenic conditions, CD34+ EPC form spindle-shaped cells, which, over time, organize in capillary-like structures. Moreover, these cells express markers specific for mature endothelial cells (EC) such as CD31, E-selectin and Tie-2.

Alternatively EPC have been isolated based on the in vitro culture of mononuclear cells on fibronectin- or gelatin-coated plates in the presence of angiogenic growth factors. Isolated adherent cells that were low density lipoprotein (LDL) positive and exhibited lectin-binding ability were called EPC. Although these cells promote angiogenesis in vivo, they have monocytic features and their angiogenicity is actually caused by their production of angiogenic factors, such as VEGF, hepatocyte growth factor (HGF), G-CSF and GM-CSF. Thus, while these LDL positive, lectin-binding cells do not directly form EC, they can modulate angiogenesis.

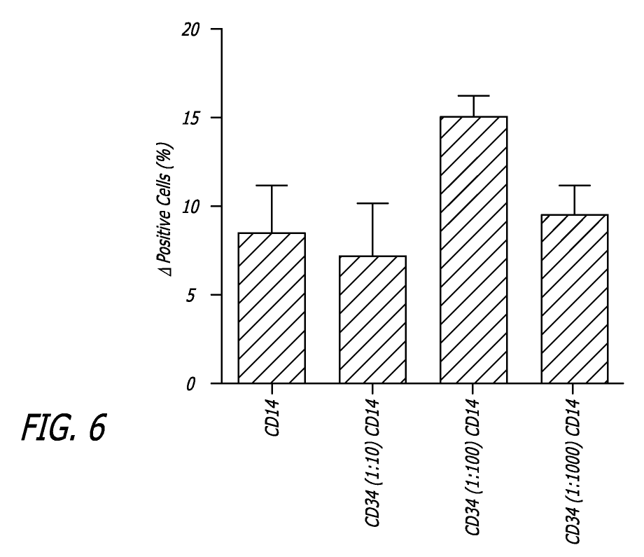

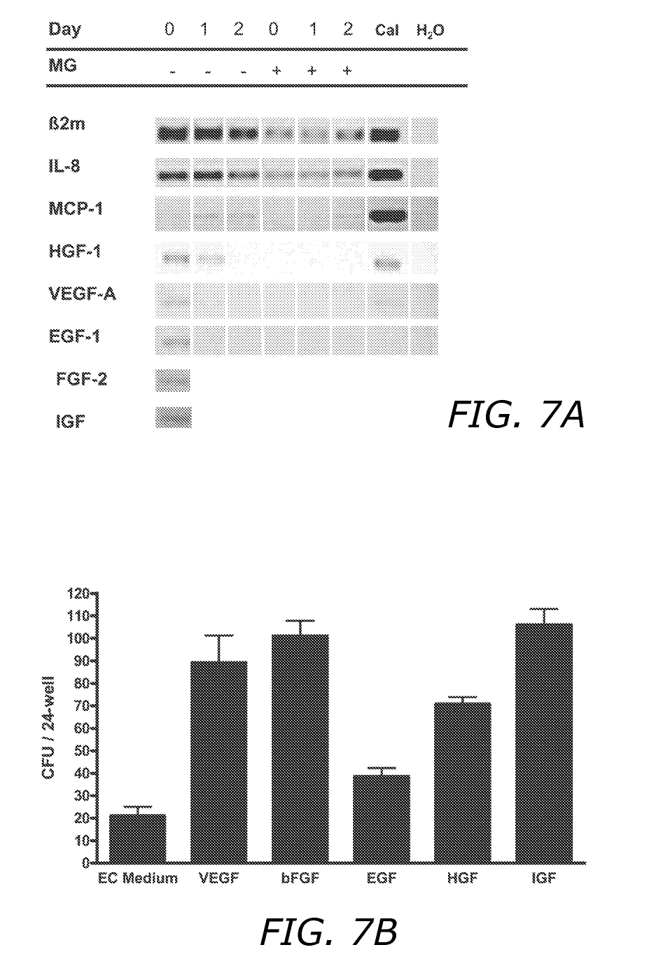

Human CD34+ EPC and CD14+ monocytes have been co-cultivated at different ratios, ranging from their physiological ratio in peripheral blood to an enriched ratio of CD34+ EPC (1:10, 1:100, and 1:1000). The CD34+ EPCs augmented endothelial cell differentiation from CD14+ monocytic cells in vitro. The CD34+ EPC not only stimulated a higher proportion of endothelial cell-like clusters, but expression of the endothelial cell markers von Willebrand Factor (vWF) and VE-cadherin is higher in co-cultured cells than in mono-cultured CD14+ cells (FIG. 6). Additionally, CD34+ EPC express pro-angiogenic genes such as EGF, HGF, VEGF-a, bFGF and IGF and IL-8 (FIG. 7a). The addition of VEGF, HGF, bFGF and IGF to CD14+ mono-cultures ameliorates CFU formation, and thus EC outgrowth, from these colonies (FIG. 7B).

The present invention therefore provides methods and compositions for inducing neovascularization in a target tissue in a subject in need thereof, comprising administering to the subject a therapeutically effective amount of expanded endothelial progenitor cells or a mixture of purified CD34+ EPCs and purified CD14+ monocytes.

In one embodiment of the present invention, a method is provided for inducing neovascularization in injured tissue in a subject by administering to a treatment site in the subject a therapeutically effective amount of a composition comprising a mixture of purified CD34+ EPCs and purified CD14+ monocytes, wherein the administering of the composition results in neovascularization in the injured tissue.

In one embodiment of the present invention, the CD34+ EPCs and CD14+ monocytes are isolated from bone marrow, from peripheral blood, or from umbilical cord blood. In one embodiment of the methods described herein, the subject is a human. In another embodiment, the cells used in the therapies are isolated from the subject's own peripheral blood.

In embodiments of the present invention, the CD34+ EPCs and CD14+ monocytes are autologous, allogenic, or HLA compatible with the subject. The number of purified CD34+ EPCs and purified CD14+ monocytes administered to a subject needing neovascularization will vary according to the severity of the injury, the size of the tissue that is ischemic, and the method of delivery. In one embodiment, the therapeutically effective amount of a composition comprising a mixture of purified CD34+ EPCs and purified CD14+ monocytes is a safe and effective amount. In another specific embodiment, the total number of cells implanted is at least 1×10⁴ cells. In another embodiment, the amount of a composition comprising a mixture of purified CD34+ EPCs and purified CD14+ monocytes administered to the subject is between about 10⁴ and about 5×10⁸ cells. The amount of cells administered to the subject will depend on the mode of administration and the site of administration. For example, a therapeutically effective cell dose via intracoronary injection (or intra-renal or intra-carotid) may be lower than that for intra-femoral injection.

The ratio of the purified CD34+ EPCs to purified CD14+ monocytes in the mixture can be, for example, from about 1:1 to about 1:10000. Alternatively, the ratio can be, in non-limiting examples, from about 1:50 to about 1:5,000, from about 1:100 to about 1:2,000, from about 1:500 to about 1:1,000, from about 1:10 to about 1:1000, from about 1:10 to about 1:1000.

In another embodiment, a method for inducing neovascularization in injured tissue in a subject is provided comprising obtaining peripheral blood from the subject, individually isolating purified CD34+ EPCs and purified CD14+ monocytes from the peripheral blood, co-culturing the purified CD34+ EPCs and purified CD14+ monocytes in a culture medium for up to four weeks to yield a population of co-cultured EPCs and administering a therapeutically effective amount of the co-cultured EPCs to a treatment site, thereby inducing neovascularization in the injured tissue.

The ratio of the purified CD34+ EPCs to purified CD14+ monocytes in the co-culture can be, for example, from about 1:1 to about 1:10000. Alternatively, the ratio can be, in non-limiting examples, from about 1:50 to about 1:5,000, from about 1:100 to about 1:2,000 or from about 1:500 to about 1:1,000, from about 1:10 to about 1:1000, from about 1:10 to about 1:1000.

Purified CD34+ EPCs and purified CD14+ monocytes are cultured under conditions favorable to survival of EPCs. In one embodiment, the culture medium contains one or more than one growth factor selected from the group consisting of vascular endothelial growth factor and basic fibroblast growth factor. In one non-limiting example, the culture medium (GMX) consists of RPMI 1640, supplemented with 20% Fetal Calf Serum, 5 U/mL heparin, 2 mM L-glutamine 1% Penicillin/Streptomycin, endothelial cell growth factor (5 μg/mL), VEGF-A (1 ng/mL) and bFGF (10 ng/mL). The CD34+ and CD14+ cells can be co-cultured in medium containing serum from other sources including, but not limited to, human serum, autologous human serum, etc. The cells are co-cultured for up to about four weeks, alternatively, for about one week, for about two weeks or for about three weeks. At the end of the co-culture period the cells are washed and prepared for administration to a patient or cryopreserved according to established protocols known to persons of ordinary skill in the art.

In embodiments of the present invention, the co-cultured EPCs are autologous, allogenic, or HLA compatible with the subject. The number of co-cultured EPCs administered to a subject needing neovascularization will vary according to the severity of the injury, the size of the tissue that is ischemic, and the method of delivery. In one embodiment, the therapeutically effective amount of co-cultured EPCs is a safe and effective amount. In another specific embodiment, the total number of expanded EPCs implanted is at least 1×10⁴ cells. In another embodiment, the amount of co-cultured EPCs administered to the subject is between about 10⁴ and about 5×10⁸ cells. The amount of co-cultured EPCs administered to the subject will depend on the mode of administration and the site of administration. For example, a therapeutically effective cell dose of co-cultured EPCs via intracoronary injection (or intra-renal or intra-carotid) may be lower than that for intra-femoral injection.

In particular embodiments of the present invention, administering can comprise an infusion of cells into the subject wherein the cells migrate to the treatment site. The infusion can comprise a systemic infusion of cells into the subject, or it can comprise an infusion of cells in the proximity to the treatment site, so as to facilitate the migration of cells to the tissue in need of vascularization. The infusion can also be performed on the blood vessels that supply blood to the target tissue, or to blood vessels which remove blood from the target tissue. In additional embodiments, the infusion of cells into the subject can comprise an intra-arterial infusion, an intramuscular infusion, an intracardiac infusion, and intracoronary infusion or an intravenous infusion. In one embodiment, the co-cultured EPCs or mixture of purified CD34+ EPCs and purified CD14+ monocytes are administered to the subject by infusion into at least one coronary artery. In another embodiment, the coronary artery is an epicardial vessel that provides collateral blood flow to the ischemic myocardium in the distribution of a chronic totally occluded vessel.

In another embodiment, a composition for inducing neovascularization in a subject is provided. The composition comprises a population of EPCs, wherein the population of EPCs comprises co-cultured EPCs or a mixture of purified CD34+ EPCs and purified CD14+ monocytes, and at least one pharmaceutically acceptable carrier.

One embodiment of the present invention provides a composition for the induction of neovascularization in a subject wherein the composition comprises a mixture of cells comprising purified CD34+ EPCs and purified CD14+ monocytes at a ratio of between about 1:1 and about 1:1000.

In some embodiments of the compositions provided herein, the compositions are provided frozen or cryopreserved and are thawed before use.

In one embodiment of the present invention, the cells which are to be administered to the subject are administered in a buffer, such as, without limitation, a saline buffer. In one preferred embodiment, the buffer comprises human blood serum isolated from the same subject who is the recipient of the therapy. Human serum can be isolated using standard procedures known to those of ordinary skill in the art. A solution comprising human blood serum can also be used to thaw a sample of cells that has been cryopreserved. In some embodiments, the solution comprising human serum comprises between about 1-20% human serum, or more preferably between about 5-15%.

In another embodiment of the present invention, the composition further comprises a highly enriched human serum cocktail which consists of a plurality of growth factors and cytokines derived from activated autologous human platelet rich plasma as generated by the Medtronic Magellan® Platelet Isolation System or similar functioning devices.

The therapeutically effective amount of the co-cultured EPCs or mixture of purified CD34+ EPCs and purified CD14+ monocytes can be suspended in a pharmaceutically acceptable carrier or excipient. Such carriers include, but are not limited to, basal culture medium plus 1% serum albumin, saline, buffered saline, dextrose, water, biodegradable biocompatible matrices, and combinations thereof. Examples of biodegradable biocompatible matrices include, but are not limited to, solubilized basement membrane, autologous platelet gel, collagen gels or collagenous substrates based on elastin, fibronectin, laminin, extracellular matrix and fibrillar proteins, alginates, chitosans, and synthetic compositions such poly lactic acid, poly glycolic acid, polyethylene oxide, polyethylene glycol, etc. The formulation should suit the mode of administration. Accordingly, the invention provides a use of endothelial producing cells, such as co-cultured EPCs or a mixture of purified CD34+ EPCs and purified CD14+ monocytes, for the manufacture of a medicament to induce neovascularization in a target tissue in a subject in need thereof. In some embodiments, the medicament further comprises growth factors, chemokines or cytokines.

In one embodiment, the composition is formulated in accordance with routine procedures as a pharmaceutical composition adapted for intravenous administration to human beings. Typically, compositions for intravenous, intra-arterial or intracardiac administration are solutions in sterile isotonic aqueous buffer. Where necessary, the composition can also include a local anesthetic to ameliorate any pain at the site of the injection.

A variety of means for administering cells to subjects will, in view of this specification, be apparent to those of ordinary skill in the art. Such methods include injection of the cells into a target site in a subject. Cells can be inserted into a delivery device which facilitates introduction by injection or implantation into the subjects. Such delivery devices can include tubes, e.g., catheters, for injecting cells and fluids into the body of a recipient subject. In a preferred embodiment, the tubes additionally have a needle, e.g., a syringe, through which the cells of the invention can be introduced into the subject at a desired location. In one embodiment, cells are formulated for administration into a blood vessel via a catheter (where the term “catheter” is intended to include any of the various tube-like systems for delivery of substances to a blood vessel). The cells can be prepared for delivery in a variety of different forms. For example, the cells can be suspended in a solution or gel. Cells can be mixed with a pharmaceutically acceptable carrier or diluent in which the cells of the invention remain viable. Pharmaceutically acceptable carriers and diluents include saline, aqueous buffer solutions, solvents and/or dispersion media. The use of such carriers and diluents is well known in the art. The solution is preferably sterile and fluid, and will often be isotonic.

Modes of administration of the co-cultured EPCs, and the mixture of purified CD34+ EPCs and purified CD14+ monocytes include but are not limited to systemic intracardiac, intracoronary, intravenous or intra-arterial injection and injection directly into the target tissue at the intended site of activity. The preparation can be administered by any convenient route, for example by infusion or bolus injection and can be administered together with other biologically active agents. The co-cultured EPCs, CD34+ EPCs and the CD14+ monocytes, when administered, migrate or home to the target tissue in response to chemotactic factors produced due to injury or disease.

In one embodiment of the methods described herein, a bioactive agent is administered to the subject in combination with the administration of cells. The bioactive agent can be administered to the subject before, concurrently, or after the administration of the cells. In one preferred embodiment, the bioactive promotes angiogenesis, neovasculogenesis, or both. In another embodiment, the bioactive promotes the proliferation or differentiation of the EPCs. In one embodiment, the bioactive agent is VEGF or bFGF or a fragment thereof which retains a therapeutic activity.

In one embodiment of the present invention, the subject needing neovascularization of an injured tissue suffers from ischemia. The ischemia can be selected from the group consisting of limb ischemia, chronic myocardial ischemia, ischemic cardiomyopathy, myocardial ischemia, cerebrovascular ischemia, renal ischemia, pulmonary ischemia and intestinal ischemia. The methods described herein are not limited to ischemia in any particular tissue, but are applicable to any type of ischemia. For example, in one embodiment, the subject suffers from ischemia in multiple tissues. In this instance, a systemic infusion of cells to the subject can be performed, or alternatively or in combination, one or more localized infusions near the ischemic tissue can be performed. Any method currently available for delivering cells to a subject can be used to administer cells to a subject in the methods described herein.

Some embodiments of the present invention provide methods for inducing neovascularization in a subject in need thereof. There are numerous conditions that cause the necessity of a mammal to be in need of neovascularization. For example, the mammal can have a wound that requires healing. The wound can be an acute wound, such as those caused by burns and/or contact with hard and/or sharp objects. For example, patients recovering from surgery, such as cardiovascular surgery, cardiovascular angioplasty, carotid angioplasty, and coronary angioplasty all require neovascularization. The wound can also be a chronic wound. Some examples of chronic wounds include ulcers, such as vascular ulcers and diabetic ulcers. Inducing neovascularization from the cells described in the present invention is especially effective in increasing cardiac or peripheral (i.e. limb) vascularization. Therefore, the method is especially effective in treating cardiac and peripheral ischemia.

In particular, the present invention methods are useful for neovasculogenesis for the treatment of myocardial ischemia in humans. Administration of co-cultured EPCs or a mixture of purified CD34+ EPCs and purified CD14+ monocytes according to the methods of the present invention can be used as a sole treatment or as an adjunct to surgical and/or medical treatment modalities. For example, the methods described herein for treatment of myocardial ischemia can be used in conjunction with coronary artery bypass grafting or percutaneous coronary interventions. The methods described herein are particularly useful for subjects that have incomplete revascularization of the ischemic area after surgical treatments and, therefore, have areas of ischemic but viable myocardium. Subjects that can significantly benefit from the neovasculogenesis according to the methods of the invention are those who have large areas of viable myocardium jeopardized by the impaired perfusion supplied by vessels that are poor targets for revascularization techniques. Other subjects that can benefit from the therapeutic vasculogenesis methods are those having vessels of small caliber, severe diffuse atherosclerotic disease, and prior revascularization, in particular bypass grafting.

In one embodiment, the composition fulfills a paracrine function, such that the factors that are released as a result of the synergistic interplay between the two cell types are capable of inducing one or more downstream events, such as, but not limited to, stem cell recruitment or mobilization, wound healing, tissue remodeling, neovascularization, tissue repair or regeneration.

Apart from delivering the compositions via different catheter or syringe-based approaches, the paracrine role of the compositions can be accomplished by incorporating them into implantable medical devices including, but not limited to, patches, scaffolds, or the like, made of biocompatible matrices that are then placed over the tissue or organ of interest.

Additionally, the compositions of the present invention could be used to seed or cover, and in the process pacify, synthetic vascular grafts prior to implantation.

Similarly, autologous or allogenic tissue grafts, such as heart valves, valve leaflets, etc., can be seeded or pacified prior to implantation.

Additionally, the compositions of the present invention are suitable, together with other primary cell types such as, but not limited to, skeletal myoblasts, mesenchymal stem cells, adipose-tissue derived stem cells, embryonic stem cells, etc. to provide essential survival factors for the primary cells immediately following transplantation.

Although the expanded EPCs or mixture of purified CD34+ EPCs and CD14+ monocytes can be injected directly into the area of the injured tissue, they can also be infused into an artery supplying the area of ischemia or injury.

These examples are meant to illustrate one or more embodiments of the present invention and are not meant to limit the invention to that which is described below.

### Example 1

### Isolation of CD34+ and CD14+ Cells

Mononuclear cells (MNC) were isolated from heparinized blood by lymphoprep density gradient centrifugation (Nycomed, Oslo, Norway). Total MNC were stained with a monoclonal antibodies (moAbs) against CD14 (monocytes, IQ Corp., Groningen, The Netherlands) and/or CD34 and isolated by magnetic cell sorting (MACS). CD34+ MNC were further purified by fluorescence activated cell sorting (FACS) to yield purified CD34+ cells. CD14+ cells were labelled with CM-Dye I, a red fluorescent dye, that labels proteins in the cytosol and that is used to distinguish cells in a mixed population of cells.

### Example 2

### Co-Cultivation of CD34+ and CD14+ Cells

Purified CD14+ and CD34+ were cultured either alone or in co-culture at ratios of CD34:CD14 cells of 1:1, 1:10, 1:1000 in GMX media. GMX consists of RPMI 1640, supplemented with 20% Fetal Calf Serum, 5 U/mL heparin, 2 mM L-glutamine 1% Penicillin/Streptomycin, endothelial cell growth factor (5 μg/mL), VEGF-A (1 ng/mL), bFGF (10 ng/mL) for four weeks.

Additional culture additive suitable for use in producing co-cultured EPCs include, but are not limited to, serum replacers, autologously derived human serum, And recombinant growth factor mixtures.

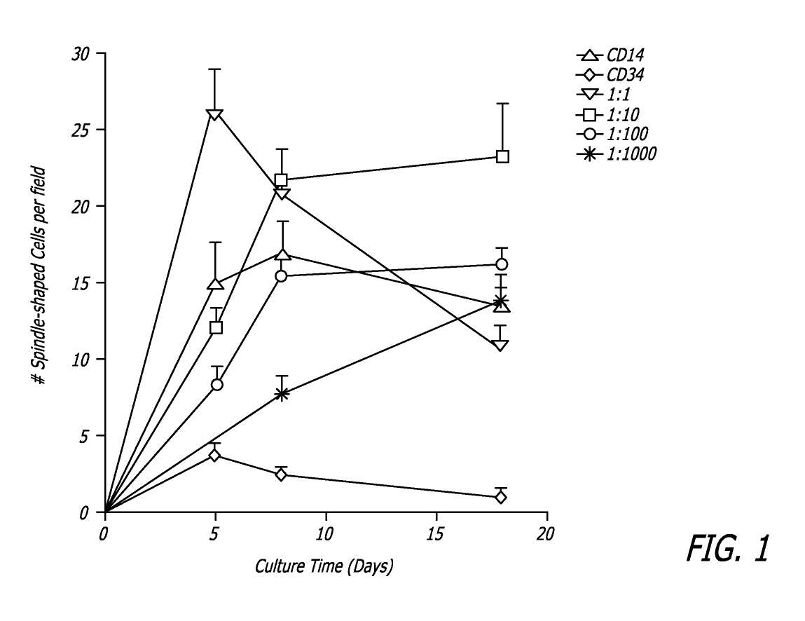

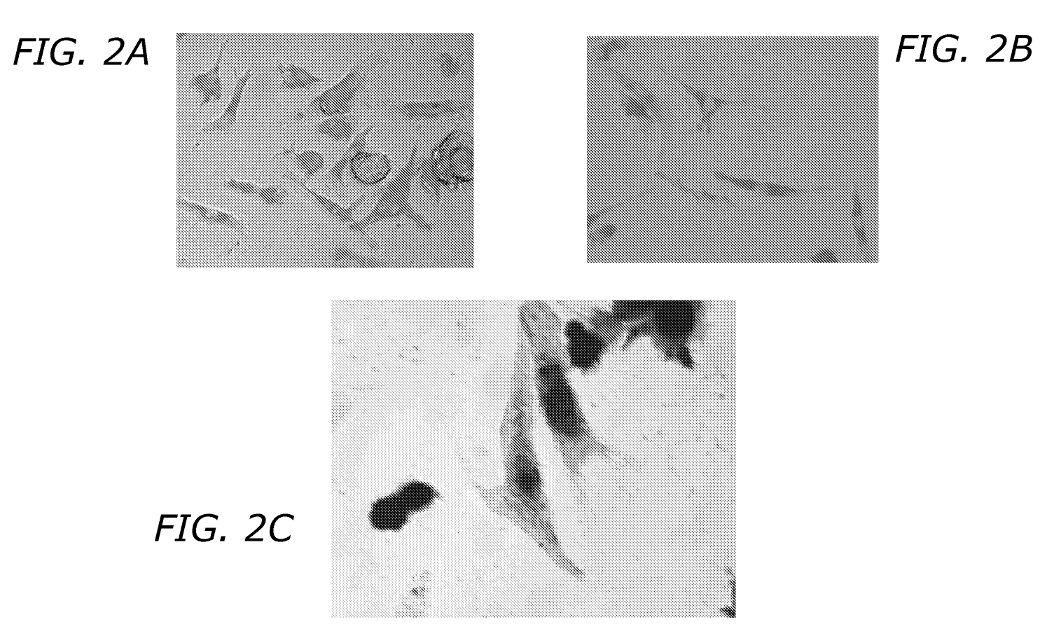

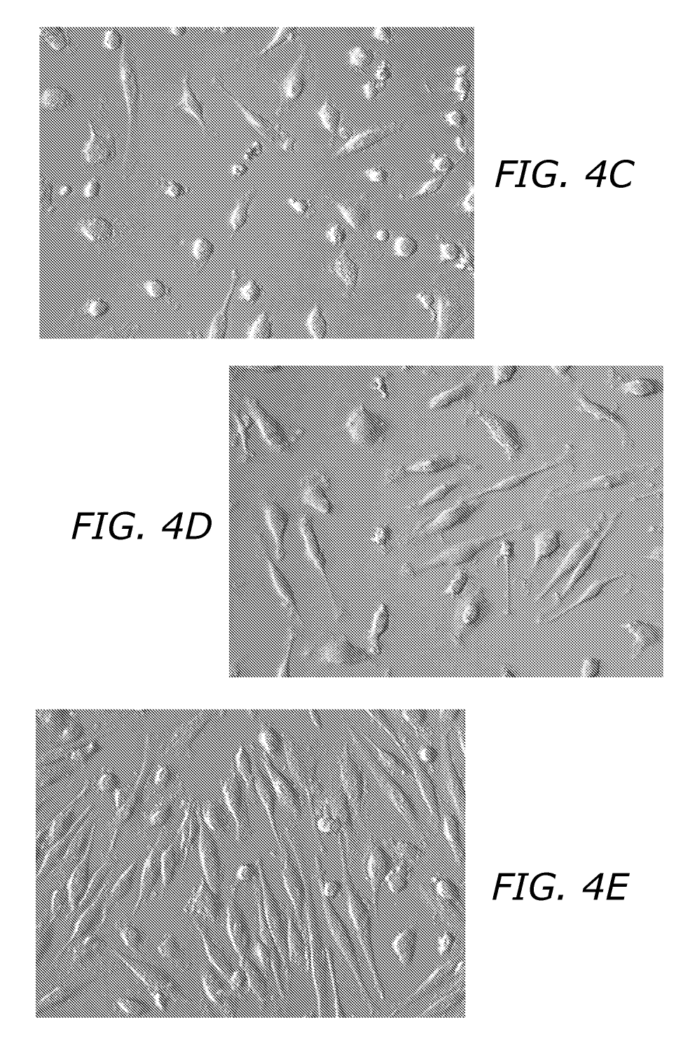

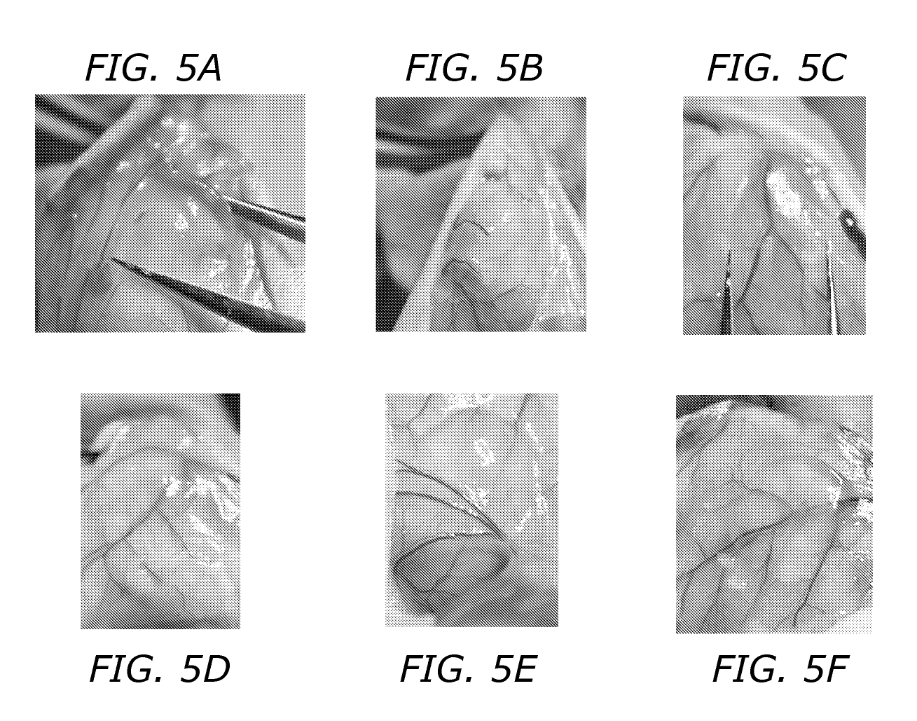

After four weeks in culture, the number of spindle-shaped cells, representing cells which have the ability to form new blood vessels, were determined. The number of spindle-shaped cells was increased in the co-cultures over solo cultures of either CD14+ or CD34+ cells alone (FIGS. 1 and 4). Differences in numbers of spindle shaped cells are clearly observed. The combination of CD34:CD14 results in higher numbers of spindle-shaped cells than monoculture of CD34+ or CD14+ cells. The number of spindle-shaped cells was highest in the co-culture at a CD34:CD14 ratio of 1:1000. Furthermore, the spindle-shaped cells are CD14+ cell-derived since the CD14+ cells were labeled red with CM-Dil prior to culture, and the resultant spindle-shaped cells expressed the red label and the endothelial cell marker CD31 (FIG. 2A-C). FIG. 2A depicts CM-Dil (red) labeled spindle-shaped cells, derived from CD14+ cells in close contact with an unlabeled CD34+ cell. FIG. 2B: spindle shaped cells are CD14-derived as indicated by CM-Dil (red) label. FIG. 2C: Co-cultured cells express CD31, an endothelial cell marker, after 28 days of culture.

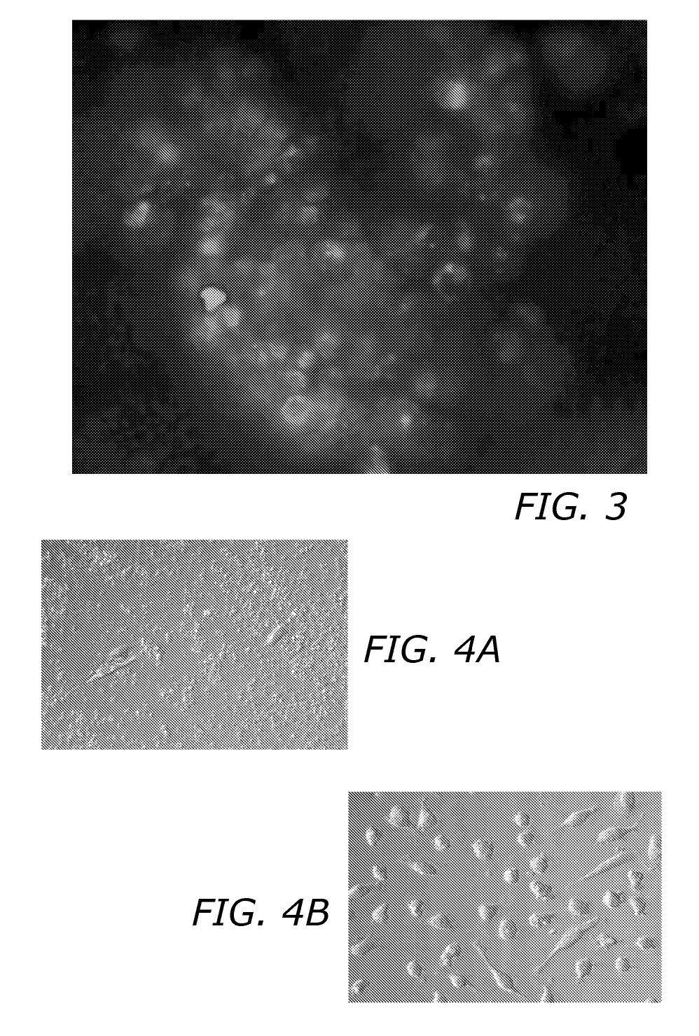

Mononuclear cells were isolated according to standard procedures and CD34+ and CD14+ cells were isolated from the MNC fraction as described. CD34+ cells were labeled green with CFSE dye and CD14+ cells were labeled red with CM-Dil. Both labeled cell fractions were mixed with remaining MNC and plated. FIG. 3 demonstrates that the CFUs that are formed (from which EC sprout) are predominantly made up from CD14+ and CD34+ cells. The cells that form EC-like cells (spindles) are predominantly CD14-derived. The CD34+ cell which lies in the centre of the CFU in FIG. 3 suggests that CD34+ cells provide the proper signals for the CD14+ cell to differentiate.

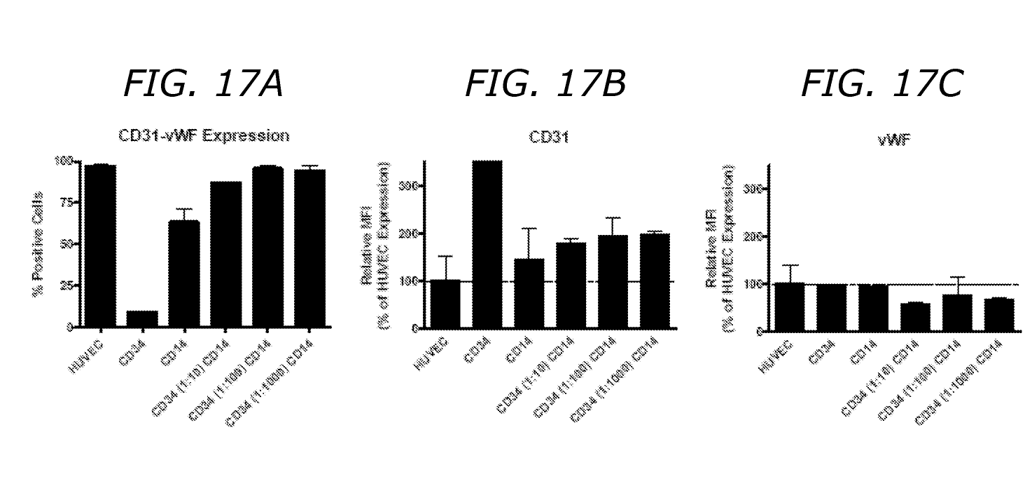

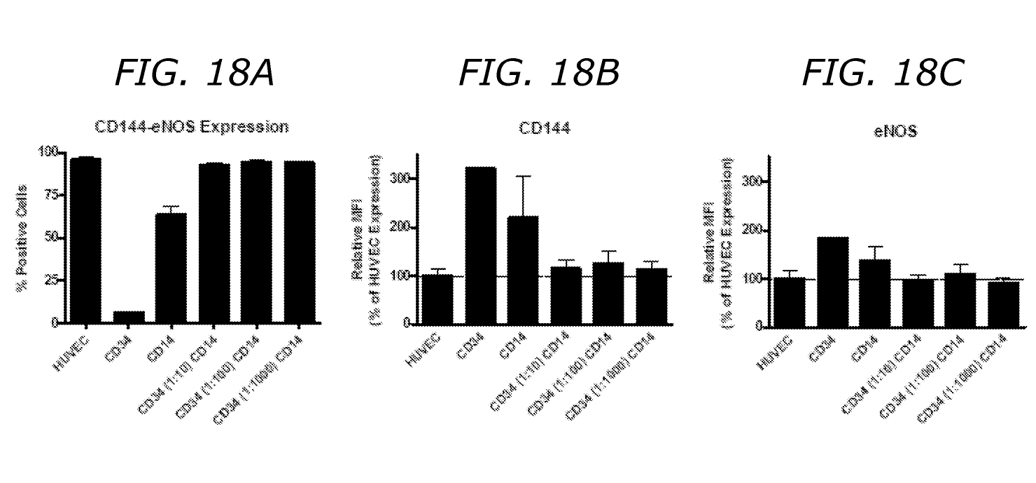

Adherent cells resulting from co-cultures were characterized for the endothelial surface markers CD31, VE-Cadherin (CD144), von Willebrand Factor (vWF) and endothelial nitric oxide synthase (eNOS). Expression of vWF and VE-cadherin are higher in co-cultivated cells than in monocultured CD14+ cells (FIG. 6). Double expression of CD31 and vWF (FIG. 17) and CD144 and eNOS (FIG. 18) was higher in co-cultures than in the monocultures and were comparable to that observed in human umbilical vein endothelial cells (HUVEC). Co-cultivation of CD34+ and CD14+ cells in all ratios resulted in higher percentages (approximately 95%) of endothelial marker expressing cells than monocultured cells.

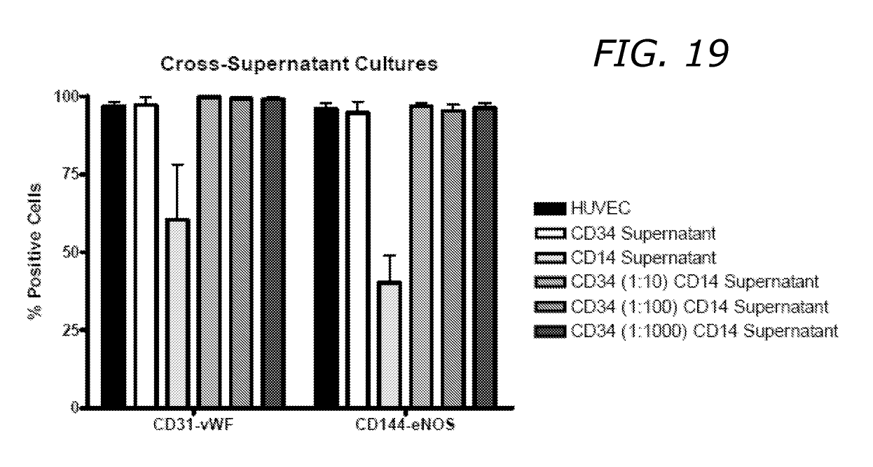

In order to determine wherein the enhanced endothelial differentiation was a result of cell-cell contact between CD34+ and CD14+ cells or could be attributed to soluble factors secreted by the cells during culture, CD14+ cells were plated alone and the cultured with supernatants from CD34+ cells alone, CD14+ cells alone, or co-cultures of CD34+ and CD14+ cells at different ratios. After three weeks the cells were removed from the culture plates and analyzed by FACS for expression of endothelial cell specific markers (CD31, vWF, CD144 and eNOS). HUVEC were used as positive controls. Incubation of CD14+ cells with supernatants from CD34+ cells alone or from co-cultures resulted in expression of endothelial markers on the CD14+ cells (FIG. 19). Addition of neutralizing antibodies against human interleukin-8 and/or monocyte chemotactic protein (MCP-1) to the CD14+ cultures had no inhibitory effect on endothelial differentiation of CD14+ cells.

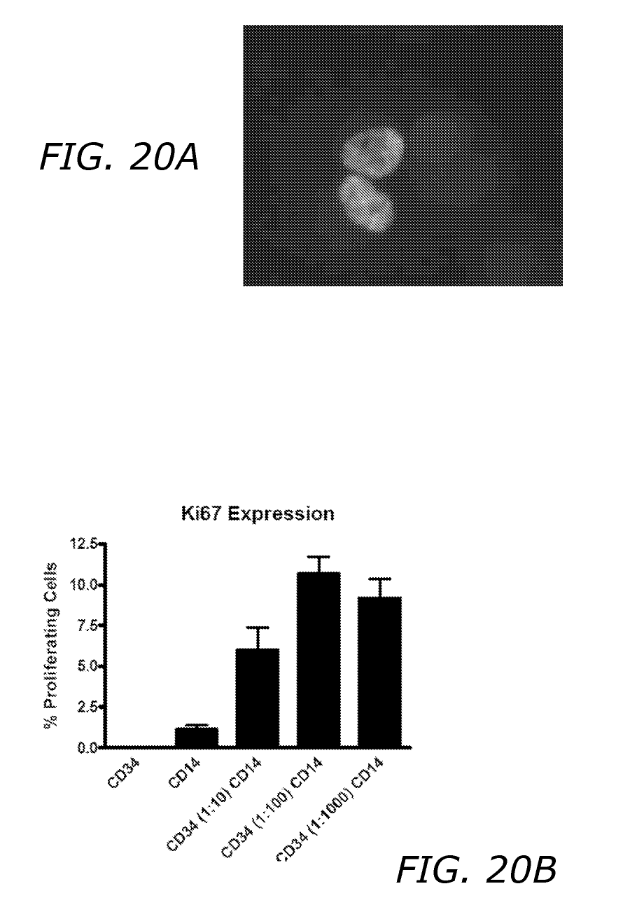

An increase in proliferating cells was seen in the CD34+/CD14+ co-cultures as evidenced by immunofluorescent staining with Ki67 (FIG. 20).

### Example 3

### Functionality of CD-14-derived Endothelial Cells

Although the newly differentiated CD14-derived endothelial cells express four distinct endothelial-specific markers, a hallmark of endothelial cell function, prevention of blood clotting, was investigated.

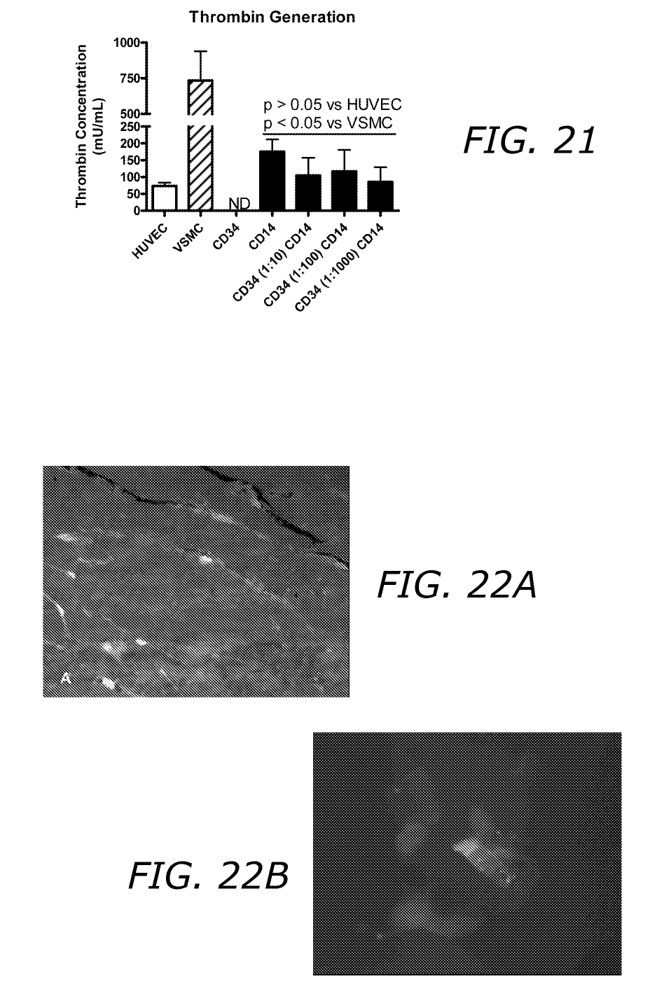

The co-cultured cells were tested in vitro for their ability to prevent thrombin generation. HUVEC were used as positive control and vascular smooth muscle cells (VSMC) were the negative control. As demonstrated in FIG. 21, HUVEC prevent thrombin generation but VSMC do not. Co-cultured CD34+ and CD14+ cells, as well as CD14+ cells cultured alone, prevent thrombin generation (FIG. 21).

### Example 4

### Effect of CD34+ Cells on Expression of Pro-Angiogenic Genes

CD34+ EPC express pro-angiogenic genes such as EGF, HGF, VEGF-a, bFGF, IGF and IL-8 (FIG. 7A) which may ameliorate expression of these genes in CD14+ cells. CD34+ produce these factors (FIG. 7A) and addition of these factors to CD14+ monoculture increase CFU formation (FIG. 7B). Addition of VEGF, HGF, bFGF and IGF to CD14+ mono-cultures ameliorates CFU formation, and thus EC outgrowth, from these colonies (FIG. 7B).

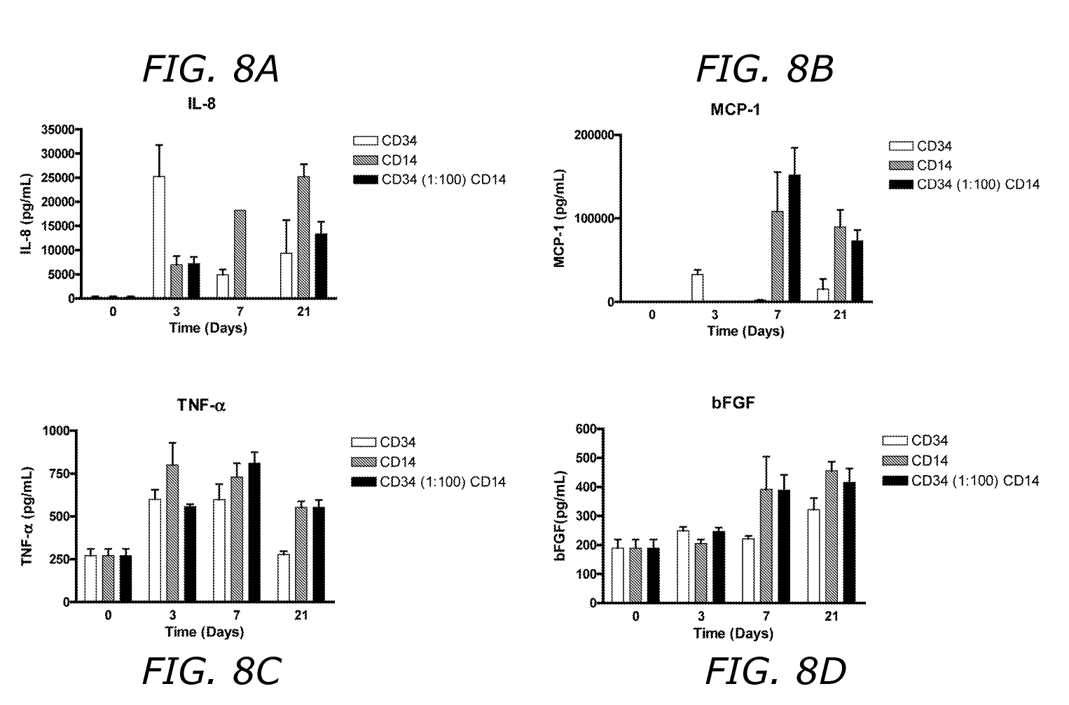

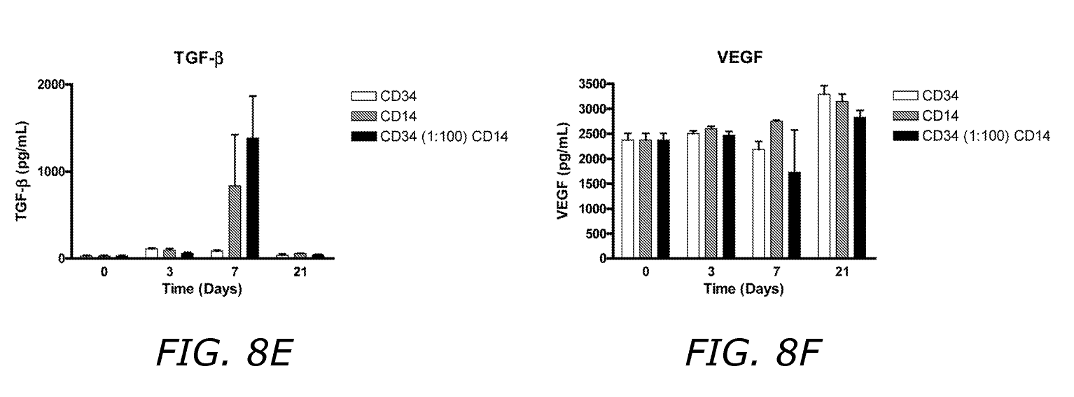

Additionally, cell populations (CD34+ alone, CD14+ alone, and CD34+:CD14+ (1:100) were cultured for up to 21 days. At days 3, 7, 21 conditioned media was removed from the test conditions and frozen for later analysis of growth factors by multiplex arrays as depicted in FIG. 8. The readings at time 0 represents the basal conditions and represent the composition of the growth media that was used for all three test conditions.

IL-8 is predominantly produced by the CD34+ cells early in culture. From day 7 on, the CD14+ cells take over the production of IL-8 (FIG. 8A). Additionally, MCP-1 is produced exclusively by CD34+ at the early time point day 3 and from day 7 on, this is also taken over by CD14+ cells (FIG. 8B). Together, this switch in source of growth factors from CD34+ cells to CD14+ cells suggests that in vivo, CD34+ cells produce IL-8 and MCP-1, resulting in the recruitment of CD14+. Upon recruitment, these CD14+ cells enter an amplifying loop in which the CD14+ cells produce significant amounts of IL-8 and MCP-1, resulting in increased recruitment of CD14+ cells.

TNF-alpha is produced in similar amounts by CD34+ and CD14+ cells up to day 21 (FIG. 8C). Expression of TNF-alpha is an important feature of endothelial cells (EC). These results suggest that the EC formed in the co-cultures possess functional properties, an observation further supported by their expression of vWF.

The data also reveals the presence of pro-angiogenic bFGF in the culture supernatant from day 7 to 21 (FIG. 8D). This may result in the formation of a pro-angiogenic niche in which recruited CD14+ enter a pro-angiogenic environment and differentiate towards EC.

TGFb is upregulated at day 7 only (FIG. 8E). TGFb plays a role in the maturation of EC. Interestingly, spindle-shaped cells, which have the morphology of EC, are seen for the first time around day 7.

VEGF concentrations remain constant throughout the culture period, suggesting that VEGF is consumed, but also replaced by the cells (FIG. 8F).

### Example 5

### Blood Vessel-Forming Activity of CD34+ and CD14+ Cells

Purified CD34+ cells, purified CD14+ cells or mixtures of CD34:CD14 cells at ratios of 1:10, 1:100, 1:1000, were mixed with 200 μL Matrigel® that was supplemented with 10 ng basic fibroblast growth factor (b-FGF, Chemicon, Temecula, Calif.) and 12 U heparin (Leo Pharma, Ballerup, Denmark) at 10,000 cells per implant and implanted subcutaneously in nude mice. Bare Matrigel® contained the b-FGF and heparin supplement. A total of four samples were implanted in each animal comprising two bare Matrigel® controls and two test samples.

TABLE 1

| Group | Cells | N |
|---|---|---|
| 1 | CD34 | 6 |
| 2 | CD14 | 6 |
| 3 | CD34:CD14 = 1:10 | 6 |
| 4 | CD34:CD14 = 1:100 | 6 |
| 5 | CD34:CD14 = 1:1000 | 6 |

After 14 days, the Matrigel® pellets were explanted, partly snap-frozen in liquid nitrogen for immunohistochemistry, or fixed in 2% paraformaldehyde in 0.1 M sodium phosphate buffer, dehydrated and embedded in resin (Technovit 8100, Heraeus Kulzer, Wehrheim, Germany). For overall morphologic evaluation, 2 μm sections of resin-embedded Matrigel® pellets were stained with toluidin blue.

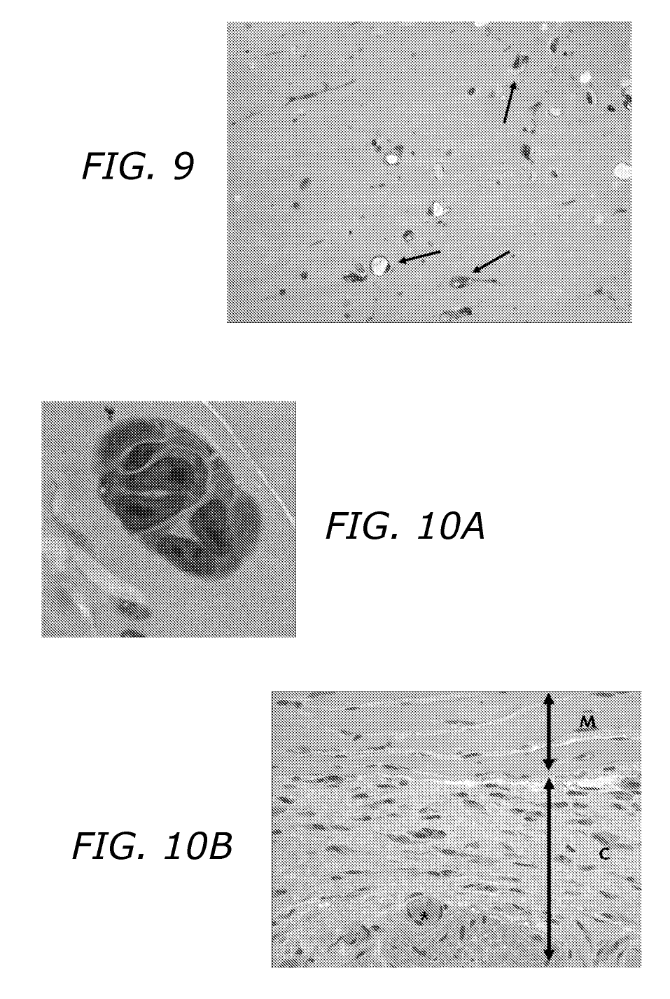

As depicted in FIGS. 9 and 10, CD34+ cell-containing Matrigel® implants (Group 1) were neovascularized by capillaries and small vessels (2-4 endothelial cells in diameter). Recruitment of inflammatory cells including monocytes, but not neutrophils, was observed. The Matrigel® implants were encapsulated with tissue containing macrophages and fibroblasts but signs of extensive inflammation were not seen.

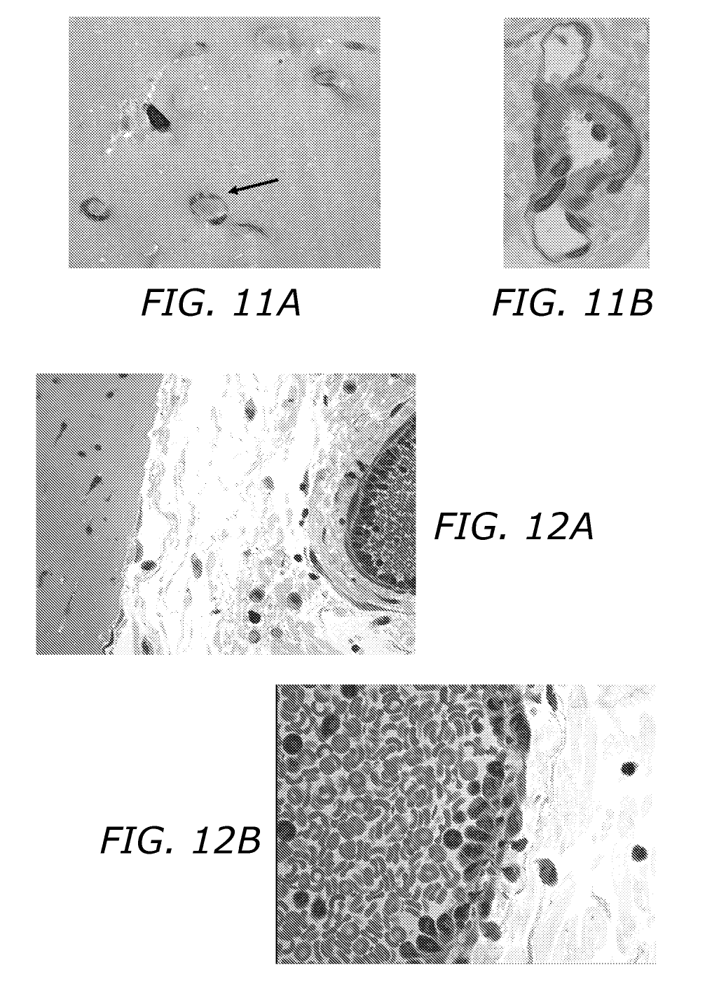

In Matrigel® implants seeded with only CD14+ cells (Group 2), similar capillaries and small vessels were seen (FIG. 11A). However, sprouting of vessels in the capsulation tissue was observed (FIG. 11B) and activation of the vasculature was evident. Extravasation of inflammatory cells and the presence of mast cells (FIG. 11C) were observed (FIGS. 12A and 12B).

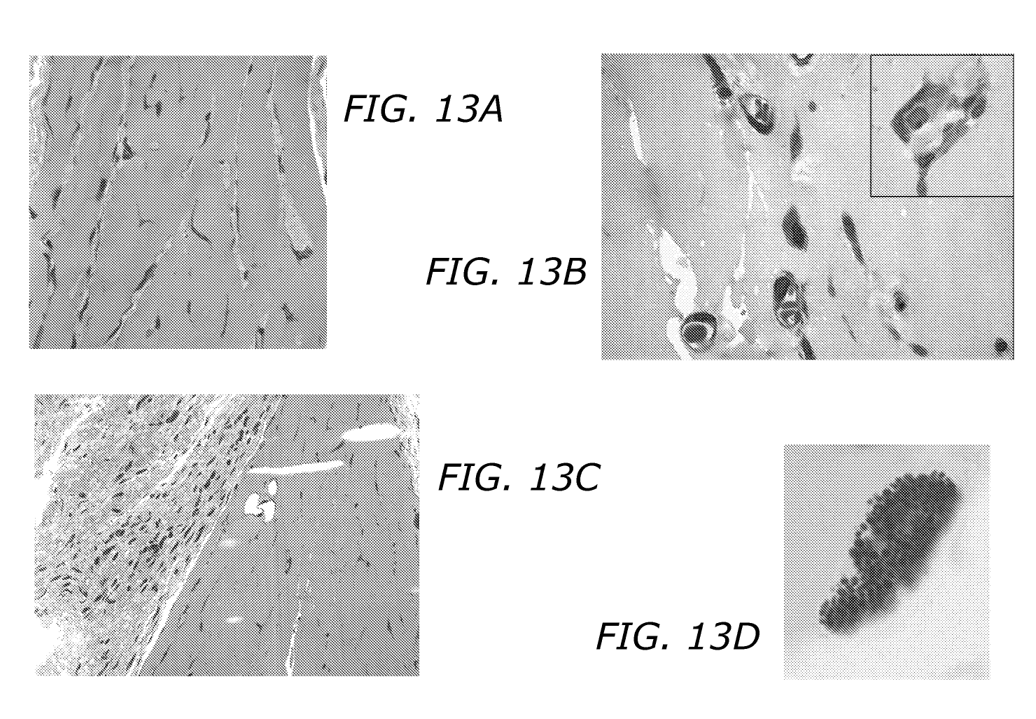

In Matrigel® implants seeded with CD34+ and CD14+ cells in a ratio of 1:10 (Group 3), vascularization of the implants was also observed (FIG. 13A). The vessels were larger and the number of functional vessels is greater (FIG. 13B) than after transplantation of CD34+ cells alone suggesting improved maturation of neovessels than after implantation of either cell type alone. The surrounding tissues was activated (FIG. 13C) and mast cells were readily detected (FIG. 13D).

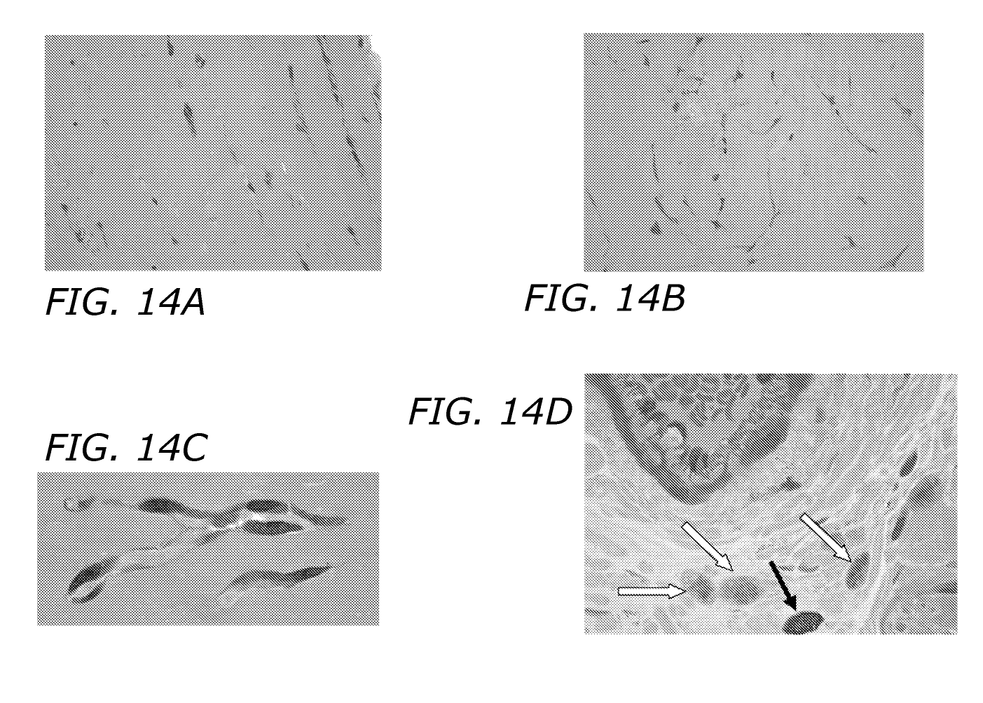

Vascular structures and activation within the capsulation tissue were also observed in Matrigel® implants seeded with CD34+ and CD14+ cells in a ratio of 1:100 (Group 4). The vessels were larger and more organized than in implants seeded with either CD34+ or CD14+ cells alone (FIGS. 14A and 14C) although more primitive vascular structures were also observed (FIG. 14B). The extent of vascularization at 1:100 ratio was less than that seen at the 1:10 ratio. Activation of the encapsulation tissue and mast cells was also present (FIG. 14D).

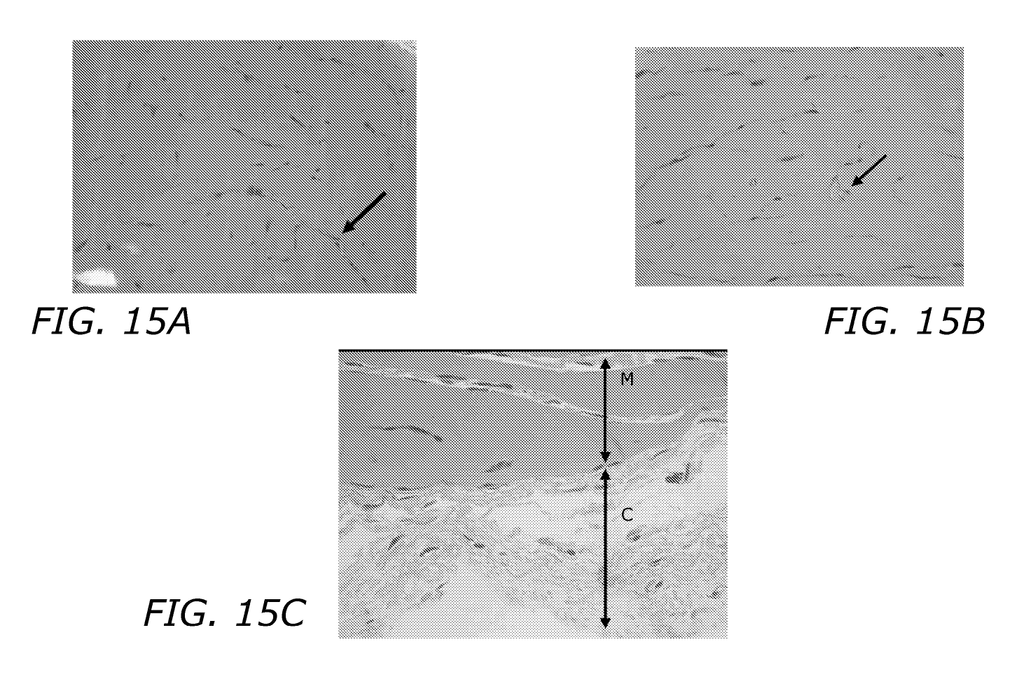

The vascular structures observed in Matrigel® implants seeded with CD34+ and CD14+ cells in a ratio of 1:1000 (Group 5) were more primitive than those observed in the other experimental groups. Although some capillaries were seen (FIG. 15A, arrow), predominantly primitive, tube-like structures were observed (FIGS. 15A and 15B). Furthermore, the extent of vascularization in the Matrigel® implant was lowest of all the experimental groups. The encapsulation tissue was relatively quiescent and comparable to that seen with implantation of CD34+ cells alone (FIG. 15C).

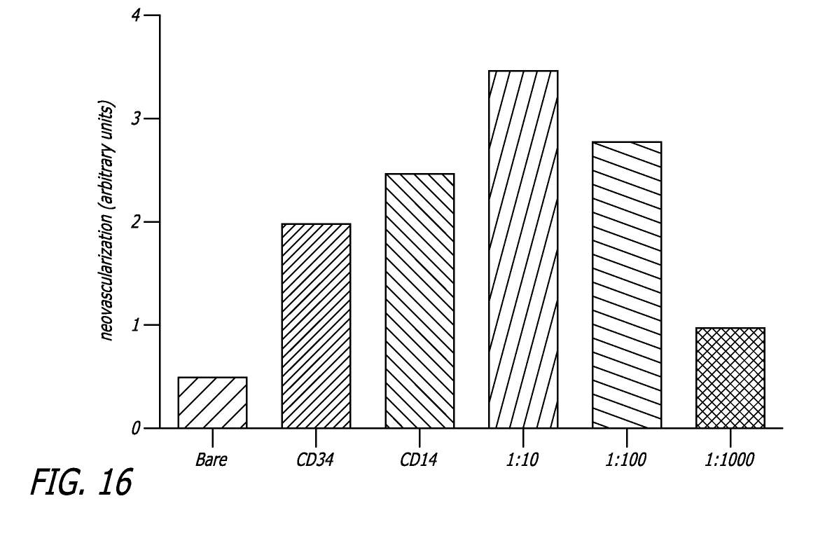

Semi-quantitative scoring of neovascularization of the Matrigel® implants is depicted in FIG. 16.

Both CD34+ and CD14+ cells alone are capable of inducing neovascularization in hypoxic Matrigel® in vivo. However, CD14+ cells induce an increased number of infiltrating cells in both the Matrigel® and the surrounding tissues than CD34+ cells including mast cells. The mast cells secrete a plethora of growth factors and chemokines and therefore the mast cells may actively contribute to recruitment of inflammatory cells and neovascularization of the Matrigel® implant.

The combination of CD34+ and CD14+ cells at a 1:10 ratio results in higher numbers of large vessels as well as a larger number of functional vessels, indicating that the combination of CD34+ and CD14+ cells promotes the maturation of the neovessels.

### Example 6

### Localization of Human and Murine Cells in Matrigel® Implants

Human cells were labeled with Dil prior to implantation and their presence in Matrigel® implants was detected with fluorescence microscopy at 14 days after implantation. In order to detect all cells present in the section, the nuclei were labeled with DAPI.

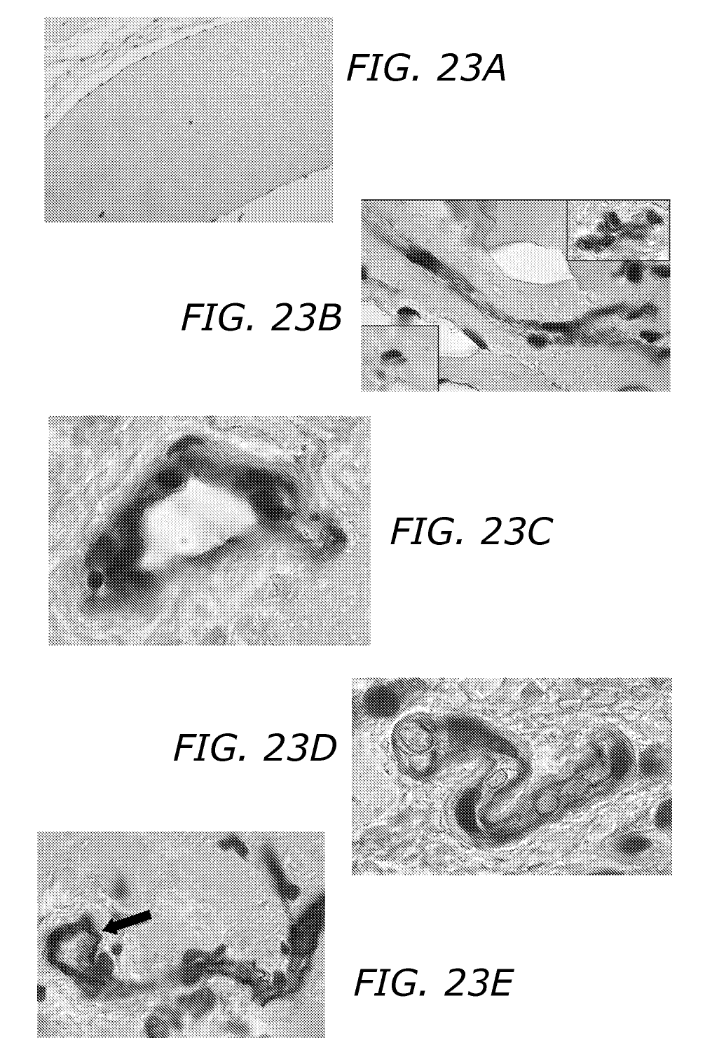

Although incorporation of human cells into the neovasculature was observed (FIG. 22), the majority of the vessels were of mouse origin. This was confirmed by immunohistochemical staining of Matrigel® sections with monoclonal antibodies to murine CD31 (FIG. 23). Both capillaries as well as larger vessels were stained with antibodies to mouse-specific markers.

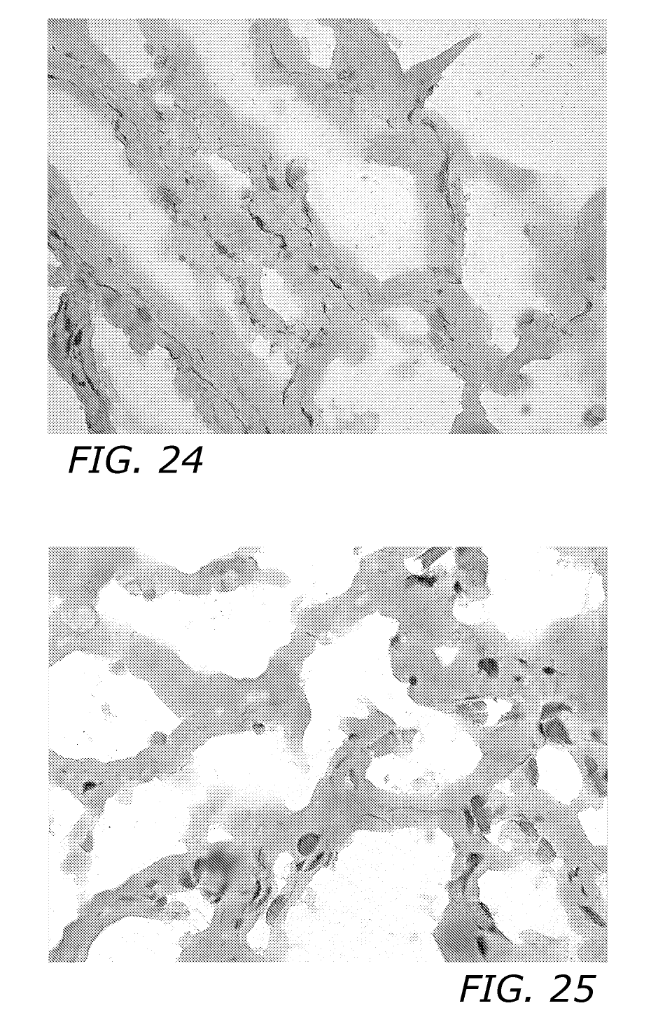

Additionally, there was an influx of murine monocytes/macrophages into the Matrigel® implant (FIG. 24). Expression of the pro-angiogenic factor vascular endothelial growth factor (VEGF) was detected in all cell-loaded Matrigel® implants compared to bare Matrigel® controls (FIG. 25).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

Certain embodiments of this invention are described herein, including the best mode known to the inventors for carrying out the invention. Of course, variations on these embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A method for inducing neovascularization in injured tissue in a subject comprising:
administering to a treatment site in said subject a therapeutically effective amount of a composition comprising a mixture of purified CD34+ endothelial progenitor cells (EPCs) and purified CD14+ monocytes, wherein said administering of said composition to a treatment site results in neovascularization of said injured tissue at said treatment site.

2. The method of claim 1, wherein said CD34+ EPCs and said CD14+ monocytes are individually or collectively isolated from peripheral blood or from bone marrow.

3. (canceled)

4. The method of claim 1 wherein said CD34+ cells and said CD14+ cells are administered at a ratio of between about 1:1 to about 1:1,000.

5. (canceled)

6. The method of claim 1 wherein said composition further comprises at least one pharmaceutically acceptable carrier.

7. The method of claim 6 wherein said pharmaceutically acceptable carrier is a scaffolding material or a biocompatible solution.

8. The method of claim 1 wherein said composition further comprises at least one bioactive agent selected from the group consisting of growth factors, chemokines, drugs and cytokines.

9. (canceled)

10. (canceled)

11. The method of claim 1 wherein said therapeutically effective amount of said composition comprises a total of between about 10⁴ and about 10⁸ cells.

12. (canceled)

13. The method of claim 1 wherein said injured tissue is ischemic tissue.

14. The method of claim 13 wherein said ischemic tissue is cardiac tissue.

15. A method for inducing neovascularization in injured tissue in a subject comprising
obtaining peripheral blood from said subject;
selecting CD34+ EPCs from said peripheral blood to generate purified CD34+ cells;
selecting CD14+ monocytes from said peripheral blood to generate purified CD14+ monocytes;
culturing said purified CD34+ cells and said purified CD14+ cells in a culture medium for up to four weeks to yield a population of co-cultured EPCS; and
administering a therapeutically effective amount of said co-cultured EPCs to a treatment site in the injured tissue of said subject, thereby inducing neovascularization of said injured tissue at said treatment site.

16. (canceled)

17. The method of claim 15 wherein said purified CD34+ EPCs and said purified CD14+ monocytes are co-cultured at a ratio of between about 1:1 to about 1:1,000.

18. (canceled)

19. The method of claim 15 wherein said co-cultured EPCs are administered in conjunction with at least one pharmaceutically acceptable carrier.

20. The method of claim 19 wherein said pharmaceutically acceptable carrier is a scaffolding material or a biocompatible solution.

21. The method of claim 15, wherein said administering step further comprises administering to said subject one or more than one bioactive agent selected from the group consisting of cytokines, chemokines, drugs and growth factors.

22. (canceled)

23. The method of claim 15 wherein said therapeutically effective amount of said co-cultured EPCs is a total of between about 10⁴ and about 10⁸ cells.

24. (canceled)

25. (canceled)

26. (canceled)

27. A composition for the induction of neovascularization in a subject, comprising:
purified CD34+ EPCs;
purified CD14+ monocytes; and
at least one pharmaceutically acceptable carrier.

28. The composition of claim 27 wherein said purified CD34+ EPCs and said purified CD14+ monocytes are isolated from peripheral blood.

29. (canceled)

30. (canceled)

31. The composition of claim 27 wherein said composition comprises said purified CD34+ EPCs and said purified CD14+ monocytes at a ratio of between about 1:1 to about 1:1,000.

32. (canceled)

33. The composition of claim 27 wherein said pharmaceutically acceptable carrier is a scaffolding material or a biocompatible solution.

34. The composition of claim 27, wherein said composition further comprises one or more than one bioactive agent selected from the group consisting of cytokines, chemokines, drugs and growth factors.
